(19) United States
(12) Patent Application Publication
(10) Pub. No.: US 2006/0188536 A1
(43) Pub. Date: Aug. 24, 2006

(54) Particles from processing of oleander leaves

(75) Inventor: Crandell Addington, San Antonio, TX (US)

Correspondence Address: T. Ling Chwang;Jackson Walker L.L.P., Suite 600, 2435 N. Central Expressway, Richardson, TX, 75080, US

(73) Assignee: Phoenix Biotechnology Inc., San Antonio, TX (US)

(21) Appl. No.: 11/340,016

(22) Filed: Jan. 26, 2006

Related U.S. Application Data
(60) Provisional application No. 60/653,210, filed on Feb. 15, 2005.

(51) Int. Cl. A01N 65/00; A61K 36/185

(52) U.S. Cl. 424/405; 424/774

(57) ABSTRACT

A method for processing oleander leaves which produces finely processed oleander particles that will yield high levels of oleandrin when subjected to extraction techniques. The method utilizes washing, drying, and passing the oleander leaves through a comminuting and dehydrating apparatus that separates the particles according to size.

### RELATED APPLICATIONS

This application claims priority to U.S. Provisional Patent Application Ser. No. 60/653,210, entitled “Particles from Processing of Oleander Leaves,” filed on Feb. 15, 2005, having Crandell Addington listed as the inventor, the entire content of which is hereby incorporated by reference.

### STATEMENT OF RIGHTS TO INVENTIONS MADE UNDER FEDERALLY SPONSORED RESEARCH

No federal funds were used in the development of the present invention.

### BACKGROUND

This invention relates to a method for processing oleander leaves which produces very fine oleander particles that are suitable for the extraction of oleandrin.

Nerium oleander is an ornamental plant widely distributed in subtropical Asia, the southwestern United States, and the Mediterranean. Its medical and toxicological properties have long been recognized. It has been used, for example, in the treatment of hemorrhoids, ulcers, leprosy, snake bites, and even in the induction of abortion. Oleandrin, an important component of oleander extract, is a potent inhibitor of human tumor cell growth. Oleandrin-mediated cell death is associated with calcium influx, release of cytochrome C from mitochondria, proteolytic processes of caspases 8 and 3, poly(ADP-ribose) polymerase cleavage, and DNA fragmentation.

One extract of the oleander plant is oleandrin. Oleandrin is a cardiac glycoside that is exogeneous and not normally present in the body. Oleandrin induces apoptosis in human but not in murine tumor cell lines (Pathak et al., Anti-Cancer Drugs, vol. 11, pp. 455463, 2000), inhibits activation of NF-kB (Manna et al., Cancer Res., vol. 60, pp. 3838-3847,2000), and mediates cell death through a calcium-mediated release of cytochrome C (McConkey et al., Cancer Res., vol. 60, pp. 3807-3812, 2000). A Phase I trial of an oleander extract has been completed recently (Mekhail et al., Am. Soc. Clin. Oncol., vol. 20, p. 82b, 2001). It was concluded that oleander extracts can be safely administered at doses up to 1.2 ml/m²/d. No dose limiting toxicities were found.

Extraction of glycosides from plants of Nerium species has traditionally been carried out using boiling water. The process of boiling water extraction of oleander (Nerium oleander) gives active components including oleandrin, nerine, and other digitoxin-like glycosides. The plant extracts are useful in the treatment of cell-proliferative diseases in animals.

U.S. Pat. No. 5,135,745 to Ozel pertains to extracts of Nerium species and their use in the treatment of cell-proliferative diseases. Ozel obtains the extraction of Nerium oleander through the use of heat. In particular, sliced plant material is placed in distilled water and boiled until an appropriate density is reached. The mixture is then filtered and heated again.

U.S. Pat. No. 5,869,060 to Selvaraj et al. pertains to extracts of Nerium species and methods of production. To prepare the extract, plant material is placed in water and boiled. The crude extract is then separated from the plant matter and sterilized by filtration. The resultant extract can then be lyophilized to produce a powder.

It is known in the art that high temperature extraction invariably destroys some, if not all, of the active components of the plants. It is clear that there is a need for a method to efficiently process leaves and extract oleandrin from the oleander plants, so that the active components of the oleander plants are not destroyed during the processing and extraction, and so that the extract may be used clinically, including in combination with radiotherapy. What is especially needed is a method for processing oleander leaves in order to obtain a suitable starting material for extraction which is efficient and provides high yields.

### SUMMARY

The present invention relates to a method for processing oleander leaves which is efficient, provides high yields, and does not utilize heat. What is produced is a very fine oleander powder which is a highly suitable starting material for further extraction of oleandrin.

An important component of the method for processing oleander leaves is the use of a patented comminuting and dehydrating system and method which utilizes vortexes of air to extract moisture and separate the plant particles by size. Suitable comminuting and dehydrating systems are described in U.S. Pat. Nos. 5,236,132; 5,598,979; 6,517,015; and 6,715,705, all to Frank Rowley, Jr., and the contents of all of these patents are hereby incorporated by reference.

In general, the method for processing oleander leaves involves collecting suitable leaves and stems, washing the collected plant material, drying the leaves and stems, and passing the leaves through an apparatus which uses vortexes of air to extract moisture and separate the plant particles by size. Larger particles are either re-processed or used as coarse material. The smallest particles are retained as fine oleander dust which can then be subjected to further extraction to obtain oleandrin.

### DETAILED DESCRIPTION OF PREFERRED EMBODIMENTS

One aspect of the present invention relates to a method for processing oleander leaves to produce oleander particles. These oleander particles may then be subjected to further processing for the extraction of oleandrin. The method for processing oleander leaves does not involve heating the leaves, so there is no risk of inactivation of any of the active substances. Furthermore, the method for processing oleander leaves is very efficient and produces high yields of particles containing very high levels of oleandrin.

A first step in the method for processing oleander leaves was the collection of the plant material. Preferably, fresh oleander stems were harvested in the morning from mature plants that had been pruned so that the new growth is lush and tender. The typical length of the stem collected was about 18 inches long. Care were taken not to harvest stems that show poor quality leaves or contained any disease or discoloration. The collected stems were placed loosely in storage containers that were clean and allowed air to circulate around the leaves. This kept heat from building up during transportation. The typical volume harvested was processed in two days or less.

A second step was washing the plant material. This was preferably done in four separate washes, all at room temperature. The first two washes were preferably done with plain tap water. The second two washes were preferably done with distilled or de-mineralized water. Each wash was done in a separate tank, and the stems were submersed under the water and agitated to loosen any foreign matter. After the agitation the stems were raised above the tank and were allowed to drip back into the tank. This step would also help keep the water cleaner in future tanks. In large batches, the water in each tank may need to be replaced if it became discolored. If the leaves were to be handled by hand, rubber gloves should be used for protection because the water containing large amounts of the sap would drip out of the stems after harvest.

After the last wash, the stems were individually inspected and any discolored leaves were removed and discarded. The stems were then be placed loosely on drying trays and allowed to drip so less time will be required during drying. Air could then be circulated through the leaves to speed up the air drying process.

The next step in the method for processing oleander leaves involved drying the leaves. The final drying step was preferably done in a heated chamber with forced air circulation and constant temperature control. The stems were placed on loose trays so the heated air could circulate around the leaves. The temperature was preferably kept below about 125° F. The humidity of the air inside the chamber could also be reduced with a dehumidifier to speed the process. Alternatively, fresh air could be added to the chamber while the moist air was discharged. A preferred time to dry the leaves was from about 12 to about 24 hours. The moisture from the stems was typically removed at a higher rate than from the stems. This actually allowed higher concentrations of soluble material to be moved to and left in the leaves.

After each batch of stems was removed from the drying chamber, the leaves were then stripped from the stems. The leaves should be brittle at this time and should break from the stem easily. The leaves were then stored in airtight and light-tight containers and in rooms that had filters to clean the air that entered into the rooms.

The final step in the method of processing oleander leaves involved passing the dried leaves through a comminuting and dehydrating apparatus. This was preferably done within about two days after the drying step. The comminuting and dehydrating apparatus was preferably that described in any of U.S. Pat. No. 5,236,132; 5,598,979; 6,517,015; or 6,715,705. The entire content of each of these patents is hereby specifically incorporated by reference. The leaves were fed into a vortex of air which both comminutes and dehydrates them.

One example of a comminuting and dehydrating apparatus is one with a body that has an inverted, coaxially shaped cavity with an open truncated lower end and an open upper end connected to a cylindrically shaped chamber. A sleeve, which extends through the chamber and into the cavity, is adjustable axially by a pair of jacks. A damper is adjustable relative to the sleeve to control air escaping from the cavity into the ambient atmosphere. A manifold with a velocity-enhancing venturi mechanism directs air from a blower tangentially into the chamber to create cochleated and resonating, oscillatory cyclonic air-flow activity. A portion of the air from the blower to the chamber is diverted through a feeder containing the material being comminuted and dehydrated. The comminuted and dehydrated material is gravitationally discharged through the cavity lower end. The body has a removable nozzle tip to extend the operational characteristics of the apparatus. Finer materials being comminuted may be gravitationally directed into the cavity through a tube and elbow arrangement spaced below a slot and gate arrangement in the damper.

A further example of a preferred comminuting and dehydrating apparatus is a system including a pair of intercoupled cyclone devices for comminuting and dehydrating, each equipped with a discharge containment unit. In this system, a blower is coupled with the primary cyclone to provide air flow, and a channel is included between the blower and the primary discharge containment unit to provide pressure equalization. An injection port is positioned remotely adjacent the discharge portion of the primary cyclone to permit injection of viscid substances into the low pressure area of the cone. The secondary cyclone device may include an exhaust filter and a meter for measuring the passage of material to be comminuted and dehydrated.

Yet another example of a preferred comminuting and dehydrating apparatus is another system utilizing a pair of cyclone structures for comminuting and dehydrating. Injection ports are positioned for injection of viscid substances directly into the low pressure region of each cone. The secondary cyclone structure is equipped with a lower exit port. A single blower is coupled with the cyclone structures to form an air flow loop from the primary cone bottom to the secondary cone top and from the secondary cone top to the primary cone top. Airflow for cycling material between the cones is controlled by feedback from moisture and particle size monitoring devices in a collection unit coupled with the secondary cone.

After the leaves had passed through the vortex they were separated into different ranges of particle sizes. As used herein, the term “fine particles” denotes particles having a range of sizes from about 10 microns to about 300 microns. Particles larger than about 1410 microns were separated and passed through the apparatus a second time. Particles between about 297 and about 1410 microns were retained for future procedures requiring coarse material. Particles that were smaller than about 297 microns were retained as fine oleander dust.

All retained material should be stored in airtight, light-tight containers in dehumidified storage areas. The fine oleander dust is preferably subjected to further procedures for the extraction of oleandrin. The current method for processing oleander leaves is particularly advantageous because it produces fine oleander particles. The extremely small size of the of the particles means that during extraction procedures, a maximum amount of both surface and internal volume of the oleander leaves is exposed to the extraction process. This provides an exponential increase in the oleandrin content that can be produced during extraction.

### EXAMPLE 1

### Processing Oleander Leaves

The procedure for processing oleander leaves was repeated several times using different amounts of starting material and producing different amounts of coarse material and fine oleander dust. The results are shown in Table 1 below.

TABLE 1

| | | | |
|---|---|---|---|
| Starting | | | |
| Material | | Coarse Material | Fine Oleander |
| (lbs.) | Dry Leaves (lbs.) | (lbs.) | Dust (lbs.) |

| | | | | |
|---|---|---|---|---|
| 1 | 254 | — | — | 8 |
| 2 | 200 | 40 | 19 | 6 |
| 3 | 215 | 52 | 23 | 7 |
| 4 | 149 | 43 | 26 | 10 |
| 5 | 99 | 26 | 5 | 12 |
| 6 | 118 | 29 | 7 | 13 |
| 7 | 217 | 55 | 12 | 25 |

### REFERENCES CITED

The following U.S. Patent documents and publications are hereby incorporated by reference.

### U.S. PATENT DOCUMENTS

U.S. Pat. No. 5,135,745 to Ozel
U.S. Pat. No. 5,236,132 to Rowley, Jr.
U.S. Pat. No. 5,598,979 to Rowley, Jr.
U.S. Pat. No. 6,517,015 to Rowley, Jr.
U.S. Pat. No. 6,565,897 to Selvaraj et al.
U.S. Pat. No. 6,715,705 to Rowley, Jr.

### OTHER PUBLICATIONS

Manna, S. K., N. K. Sah, R. A. Newman, A. Cisneros, B. B. Aggarwal, Oleandrin suppresses activation of nuclear transcription factor-kB, activator protein-1, and c-jun N-terminal kinase, Cancer Res. vol. 60 (2000) 3838-3847.
McConkey, D. J., Y. Lin, K. Nutt, H. Z. Ozel, R. A. Newman, Cardiac glycosides stimulate calcium increases and apoptosis in androgen-dependent metastatic human prostate adenocarcinoma cells, Cancer Res. vol. 60 (2000) 3807-3812.
Mekhail, T., C. Kellackey, T. Hutson, T. Olencki, G. T. Budd, D. Peereboom, R. Dreicer, P. Elson, R. Ganaphthi, R. Bukowski, Phase I study of Anvirzel in patients with advanced solid tumors, Am. Soc. Clin. Oncol. vol. 20 (2001) 82b.
Pathak, S., A. S. Multani, S. Narayan, V. Kumar, R. A. Newman, Anvirzel: an extract of Nerium oleander induces cell death in human but not murine cancer cells, Anti-Cancer Drugs vol. 11 (2000) 455463.

What is claimed is:

1. A method for processing oleander leaves to produce processed oleander particles, comprising:
harvesting oleander plant material to produce harvested oleander plant material, wherein the oleander plant material comprises one or more oleander stems having a plurality of oleander leaves;
washing the harvested oleander plant material to produce washed oleander plant material;
drying the washed oleander plant material to produce dried oleander plant material, wherein the dried oleander plant material comprises one or more dried oleander stems having a plurality of dried oleander leaves;
separating the dried oleander leaves from the dried oleander stems; and
passing the dried oleander leaves through an apparatus comprising at least one vortex of air, wherein the apparatus comminutes and dehydrates the dried oleander leaves to produce processed oleander particles.

2. The method of claim 1, wherein the washing the harvested oleander material comprises at least two washes with tap water and at least two washes with distilled or de-mineralized water.

3. The method of claim 1, wherein the drying the washed oleander plant material comprises storing the washed oleander plant material in a chamber having forced air circulation and a temperature below about 125° F.

4. The method of claim 1, wherein the drying the washed oleander plant material occurs for about 12 to about 24 hours.

5. The method of claim 1, wherein the apparatus produces processed oleander particles separated by particle size.

6. The method of claim 5, further comprising the step of separating the processed oleander particles having a size of less than about 297 microns to produce fine oleander dust.

7. The method of claim 6, further comprising the step of extracting oleandrin from the fine oleander dust.

8. The processed oleander particles produced according to claim 1.

9. The fine oleander dust produced according to claim 6.

10. A method for processing oleander leaves to produce processed oleander particles, comprising:
harvesting oleander plant material to produce harvested oleander plant material, wherein the oleander plant material comprises one or more oleander stems having a plurality of oleander leaves;
washing the harvested oleander plant material with tap water, or distilled water, or both, to produce washed oleander plant material;
drying the washed oleander plant material to produce dried oleander plant material, wherein the dried oleander plant material comprises one or more dried oleander stems having a plurality of dried oleander leaves, and wherein the drying comprises storing the washed oleander plant material in a chamber having forced air circulation and a temperature below about 125° F. for about 12 to about 24 hours;
separating the dried oleander leaves from the dried oleander stems; and
passing the dried oleander leaves through an apparatus comprising at least one vortex of air, wherein the apparatus comminutes and dehydrates the dried oleander leaves to produce processed oleander particles.

11. A method for processing oleander leaves to produce fine oleander dust, comprising:
harvesting oleander plant material to produce harvested oleander plant material, wherein the oleander plant material comprises one or more oleander stems having a plurality of oleander leaves;
washing the harvested oleander plant material to produce washed oleander plant material;
drying the washed oleander plant material to produce dried oleander plant material, wherein the dried oleander plant material comprises one or more dried oleander stems having a plurality of dried oleander leaves;
separating the dried oleander leaves from the dried oleander stems;
passing the dried oleander leaves through an apparatus comprising at least one vortex of air, wherein the apparatus comminutes and dehydrates the dried oleander leaves to produce processed oleander particles separated by particle size; and
separating the processed oleander particles having a size of less than about 297 microns to produce fine oleander dust.
